(19) United States
(12) Patent Application Publication
(10) Pub. No.: US 2022/0305151 A1
(43) Pub. Date: Sep. 29, 2022

(54) A MAN-FREE STERILIZATION SYSTEM CONTROL DEVICE, CIRCUITS, A METHOD AND CONTROL MODULES

(71) Applicant: SHENZHEN GUANKE TECHNOLOGIES CO., LTD, Shenzhen (CN)

(72) Inventors: Qing LAN, Shenzhen (CN); Ligen LIU, Shenzhen (CN); Shoubao CHEN, Shenzhen (CN); Minggui WANG, Shenzhen (CN); Mingxiang ZOU, Shenzhen (CN); Daqin HE, Shenzhen (CN)

(73) Assignee: SHENZHEN GUANKE TECHNOLOGIES CO., LTD, Shenzhen (CN)

(21) Appl. No.: 17/058,772

(22) PCT Filed: Oct. 10, 2020
(86) PCT No.: PCT/CN2020/120227

§ 371 Date: Nov. 25, 2020

(51) Int. Cl. A61L 2/10; A61L 9/20

(52) U.S. Cl. CPC A61L 2/10; A61L 9/20; A61L 2202/11; A61L 2202/14; A61L 2202/25; A61L 2209/11

(57) ABSTRACT

A man-free sterilization system control device, circuits, a method and control modules, including a switch control module, a system control module, the first control circuit, the second control circuit and the third control circuit; the system control module respectively connects with a switch control module, the first control circuit, the second control circuit and the third control circuit; the device judges whether there are people in the room in the order of operation of three control devices set indoors, at the door and outdoors, the UV light turns on when it is confirmed that there is nobody in the room, the device turns off the UV light man-freely when it is judged that there may be people in the room. With the turning-on mode and the man-free turning-off function of the device, the UV light can only be turned on for sterilization when there is nobody in the room.

### FIELD

The present invention relates to the technical field of intelligent control, particularly to a man-free sterilization system control device, circuits, a method and control modules.

### BACKGROUND

UV is of good sterilization and disinfection effects. Especially when the light of UV lights is directly used in an open space like ordinary illuminating lights, the sterilization efficiency is the highest, the utilization ratio of UV rays can be improved and the air in the space the surface of irradiated objects can be sterilized at the same time. However, UV rays of UV lights directly exposed to an open space will do damage to the human body in the sterilization environment. Therefore, for the sake of safety, it is not allowed that light of ordinary UV lights is directly exposed to an open space as ordinary illuminating lights.

UV lights used in ordinary sterilization occasions are usually used for air sterilization and disinfection. Air is inhaled into a sealed cavity by a fan and then is discharged after sterilization in the cavity. This sterilization scheme is characterized by low sterilization efficiency, small sterilization scope, high UV light cost, high energy consumption and high probability of generation of noises. In some special occasions, although UV rays are allowed to be directly exposed to an open space, the UV light should be operated by professionals to ensure that there are no people in the sterilization environment during sterilization. This sterilization method is inapplicable to ordinary sterilization occasions.

The foregoing content is only used for assisting in understanding the technical scheme of the invention, but not mean the acknowledgement of that the above content is the current technology.

### SUMMARY

The main objective of the present invention is to provide a man-free sterilization system control device, circuits, a method and control modules to solve the existing technical problem that UV lights with a low safety level cannot be directly used in ordinary sterilization occasions.

The technical problem solving thinking of the invention is as follows: the space to be sterilized is refitted into a man-free sealed sterilization space, so that UV lights can be directly exposed to the space like ordinary illuminating lights, it can be avoided that UV lights apply complex design to improve the safety level, deficiencies of existing UV lights can be overcome, such as low UV ray use ratio, high energy consumption, large noises, high cost and inflexible use.

The basic solving scheme is: three control devices are set indoors, at the door and outdoors respectively, the operator seals the space to be sterilized (i.e. indoor space) with the control device after confirming that there are no people indoors and then enable the UV light.

Specific steps include:

(1) The operator enters the indoor space (i.e. the space to be sterilized), makes an inspection tour of the internal situation of the space, confirms that there are no people in the space before enabling the first control device in the space to send the first light turning-on command to the system; if it is confirmed that there are people in the space, the UV light cannot be turned on.

(2) The operator walks out of the room and closes the door, that is the space to be sterilized (i.e. indoor space) is sealed; when the door is closed, the second control device (magnetic door contact applicable) at the door sends the second light turning-on command to the system; when the door is open, the control device at the door sends the light turning-off command to the system, preventing non-operation personnel entering the space and being damaged by the UV ray; when there are several doors indoors, all of them should be turned off before the second light turning-on command is sent to the system, but when each door is opened, the light turning-off command will be sent to the system.

(3) After the operator leaves the space to be sterilized and closes the door, the third control device outside the door is enabled to send the third light turning-on command to the system.

The operation process of three light turning-on commands is the process where the operator confirms that there are no people in the space to be sterilized. When there are no people in the space to be sterilized, the UV light is turned on. After the UV light is turned on, if somebody enters the room, the system will automatically turn off the UV light to ensure safety of the operator and other personnel. In other words, with the system of three control devices, the indoor space is refitted into a man-free occasion specially designed for sterilization of UV lights to improve the use safety of UV lights.

There is a priority level for enabling of control devices. The operation will be ineffective if the outdoor control device is enabled before the indoor control device is enabled, preventing people turning on the UV light indoors.

There are kinds of doors, such as normally closed door, normally open door or ordinary sliding door, so times of opening and closing of the door may be involved in the process where the operator walks out of the sterilization space, the control device at the door may send the light opening and closing command repeatedly, so only the order of light opening commands sent by the indoor control device and the outdoor control device is calculated when the light of the system is turned on, and the control device at the door keeps the door closed.

If only the indoor space and the door are set with a control device respectively, the indoor space can also be refitted into a man-free occasion specially for UV light sterilization, but the operator should be professional. When operating the control device in the space, the operator should take protective measures, such as wearing protective clothing or locking the indoor control device, to improve the operation safety of operators.

To achieve the aforesaid objective, the man-free sterilization system control device provided by the present invention includes: a switch control module, a system control module, the first control circuit, the second control circuit and the third control circuit; the signal input terminal of the system control module connects to the signal output terminal of the first control circuit, the signal output terminal of the second control circuit and the signal output terminal of the third control circuit, the the driving signal output terminal of the system control module connects to the driving signal input terminal of the switch control module, the switch control module is used for connecting the power and the UV light, the first control circuit is provided in the space to be sterilized, the second control circuit is provided at the door of the space to be sterilized, and the third control circuit is provided in the space to be sterilized;

the first control circuit is designed for accessing the first light turning-on signal when there is not an operator in the space to be sterilized and sending the first light turning-on signal to the system control module;

the second control circuit is designed for accessing the second light turning-on signal when the door is closed in the space to be sterilized and sending the second light turning-on signal to the system control module;

the third control circuit is designed to access the third light turning-on signal after the operator leaves the space to be sterilized and closes the door and send the third light turning-on signal to the system control module;

the system control module is used for receiving the first light turning-on signal, the second light turning-on signal and the third light turning-on signal, when the time of receiving the first light turning-on signal is earlier than that of the third light turning-on signal, turning-on drive signal is generated according to the first light turning-on signal, the second light turning-on signal and the third light turning-on signal, and the turning-on drive signal is sent to the switch control module;

the switch control module is used for receiving the turning-on drive signal and controlling the tuning-on of the UV light according to the turning-on drive signal;

the second control circuit is also used for accessing the turning-off signal when the door is opened and sending the light turning-off signal to the system control module;

the system control module is also used for receiving the light turning-off signal, generating turning-off drive signals according to the light turning-off signal and sending the turning-off drive signal to the switch control module;

the switch control module is used for receiving the turning-off drive signal and controlling the tuning-off of the UV light according to the turning-off drive signal.

Optionally, the switch control module, the system control module and the first control circuit form the independent first control module;

and/or, the third control circuit is an independent second control module.

Optionally, the switch control module is a relay;

and/or, the system control module is a single chip microcomputer control circuit;

and/or, the second control circuit is a magnetic door contact circuit device or a mechanical switch circuit, the door automatically accesses the second light turning-on signal when the door is closed, and the door automatically accesses the light turning-off signal when the door is opened.

Optionally, the first control circuit comprises one or several light turning-on key/s, the light turning-on key/s provide/s the first light turning-on signal;

when there is only one light turning-on key, this light turning-on key is used for providing the first light turning-on signal or turning-off signal;

when there are several light turning-on keys, one of these light turning-on keys is used for providing the first light turning-on signal or turning-off signal, and other light turning-on keys are used for setting the light turning-on time.

Optionally, the third control circuit comprises one or several light turning-on key/s, the light turning-on key/s provide/s the third light turning-on signal;

when there is only one light turning-on key, this light turning-on key is used for providing the third light turning-on signal or turning-off signal;

when there are several light turning-on keys, one of these light turning-on keys is used for providing the third light turning-on signal or turning-off signal, and other light turning-on keys are used for setting the light turning-on time.

Optionally, the first control circuit comprises the first indicator;

the first indicator is used for reminding the operator of that the first light turning-on signal has been sent when the first indicator goes on;

and/or, the third control circuit comprises the second indicator which is used for reminding the operator of that the third light turning-on signal has been sent when the second indicator goes on.

Optionally, the third control circuit comprises the enabling unit which enables the preset tool to make the third control circuit work normally.

Optionally, the enabling unit is a key switch, and the preset tool is a specific key.

Optionally, the third control circuit comprises a display screen;

the display screen is used for displaying the work time or the work state of the UV light.

Optionally, the system control module comprises the first extension interface unit, the third control circuit comprises the second extension interface unit, the first extension interface unit and the second extension interface unit are multi-way interfaces, the control signal terminal of the second control circuit connects to the control signal terminal of the second extension interface unit, and the first extension interface unit connects to the second extension interface unit via the control signal cable.

Optionally, the second control circuit comprises several control signal launch units which are provided in different door positions in a serial connection way;

the several control signal launch units are used for sending the second light turning-on signal to the system control module when doors corresponding to all the control signal launch units are closed;

the several control signal launch units are used for sending the light turning-off signal to the system control module when doors corresponding to any control signal launch units are open.

Optionally, the second control module comprises: the second control device, the third control device, the second signal interface, switch interface, the fifteenth resistor and the second light emitting diode;

the first terminal of the second control device connects to the first interface of the switch interface, the second terminal of the second control device connects to the fourth interface of the switch interface, the second interface of the switch interface connects to the third interface, the first interface of the switch interface also connects to the eighth interface of the second signal interface, the fourth interface of the switch interface also connects to the first interface of the second signal interface, the fifth interface and the sixth interface of the second signal interface also connect to the first terminal of the third control device, the second terminal of the third control devices is of grounding, the third interface and the fourth interface of the second signal interface connect to the cathode of the second light emitting diode, the anode of the second light emitting diode connects to the first terminal of the fifteenth resistor, the second terminal of the fifteenth resistor connects to the seventh interface of the second signal interface.

In addition, to achieve the aforesaid objective, the present invention also provides a control module which is applied in the man-free sterilization system. the control module comprises: a switch control module, a system control module, the first control circuit and the first control signal extension port, the signal input terminal of the system control module connects to the signal output terminal of the first control circuit and the signal output terminal of the first control signal extension port, the drive signal output terminal of the system control module connects to the drive signal input terminal of the switch control module, and the switch control module connects to the power supply and the UV light;

the first control circuit is designed for accessing the first light turning-on signal when there is not an operator in the space to be sterilized and sending the first light turning-on signal to the system control module;

the first control signal extension port is used for accessing the second light turning-on signal when the door of the space to be sterilized is closed and sending the second light turning-on signal to the system control module;

the first control signal extension port is designed to access the third light turning-on signal after the operator leaves the space to be sterilized and closes the door and send the third light turning-on signal to the system control module;

the system control module is used for receiving the first light turning-on signal, the second light turning-on signal and the third light turning-on signal, when the time of receiving the first light turning-on signal is earlier than that of the third light turning-on signal, turning-on drive signal is generated according to the first light turning-on signal, the second light turning-on signal and the third light turning-on signal, and the turning-on drive signal is sent to the switch control module;

the switch control module is used for receiving the turning-on drive signal and controlling the tuning-on of the UV light according to the turning-on drive signal;

the first control signal extension port is also used for accessing the turning-off signal when the door is opened and sending the light turning-off signal to the system control module;

the system control module is also used for receiving the light turning-off signal, generating turning-off drive signals according to the light turning-off signal and sending the turning-off drive signal to the switch control module;

the switch control module is used for receiving the turning-off drive signal and controlling the tuning-off of the UV light according to the turning-off drive signal.

To achieve the aforesaid objective, the man-free sterilization system control circuit provided by the present invention includes: power interfaces, a fuse tube, a voltage-dependent resistor, a rectifier bridge, a relay armature, a relay coil, the first power supply-the second power supply, the first control chip-the second control chip, the first control device, a master single chip microcomputer, the first diode-the fourth diode, a triode, the first resistor-the fourteen resistor, the first signal interface, the first capacitor-the fourth capacitor, the first capacitor-the ninth capacitor, the first inductor-the fourth inductor, the first switch-the fourth switch and the first light emitting diode;

the first interface and the second interface of the power interface connects to the first terminal of the fuse tube, the second terminal of the fuse tube connects to the first terminal of the voltage-dependent resistor and the first AC input terminal of the rectifier bridge, the third interface of the power interface connects to the voltage input terminal of the step-down rectifier unit, the third interface of the power interface connects to the second terminal of the voltage-dependent resistor and the second AC input terminal of the rectifier bridge respectively, the fourth interface of the power interface connects to the second terminal of the relay armature, the positive output terminal of the rectifier bridge connects to the first resistor and the second resistor respectively, the positive output terminal of the rectifier bridge also connects to the first terminal of the first inductor and the first terminal of the first capacitor respectively, the negative output terminal of the rectifier bridge is of grounding, the first terminal of the second capacitor connects to the second terminal of the first resistor and the second terminal of the first inductor respectively, the second terminal of the first capacitor and the second terminal of the second capacitor are of grounding, the second terminal of the first inductor connects to the first terminal of the fifth capacitor and the first terminal of the fifth resistor respectively, the first terminal of the sixth resistor connects to the second terminal of the fifth capacitor and the second terminal of the fifth resistor respectively, the second terminal of the sixth resistor connects to the cathode of the first diode, the second terminal of the first inductor connects to the first terminal of the second inductor, the fifth-eighth interfaces of the first control chip connect to the anode of the first diode and the second terminal of the second inductor, the second terminal of the second resistor connects to the first terminal of the third resistor, the second terminal of the third resistor connects to the first terminal of the fourth resistor, the first interface of the first control chip connects to the cathode of the second diode and the first terminal of the third capacitor respectively, the second terminal of the fourth resistor connects to the first terminal of the third capacitor, the anode of the second diode connects to the first terminal of the third inductor and the first terminal of the seventh resistor respectively, the second terminal of the seventh resistor connects to the first terminal of the eighth resistor and the first terminal of the sixth capacitor, the second terminal of the third inductor and the second terminal of the eighth resistor are of grounding, the third interface of the first control chip connects to the first terminal of the sixth capacitor, the second terminal of the third capacitor and the second terminal of the sixth capacitor are of grounding, the second interface of the first control chip is of grounding, the fourth interface of the first control chip connects to the first terminal of the ninth resistor, the second terminal of the ninth resistor connects to the first terminal of the seventh capacitor, the second terminal of the ninth resistor is of grounding, the second terminal of the seventh capacitor connects to the first terminal of the fourth inductor, the first terminal of the fourth inductor is of grounding, the second terminal of the fourth inductor connects to the anode of the third diode, the cathode of the third diode connects to the first terminal of the fourth capacitor and the first terminal of the tenth resistor respectively, the cathode of the third diode also connects to the first power supply, the second terminal of the fourth capacitor and the second terminal of the tenth resistor are of grounding;

the first terminal of the first control device connects to the first light turning-on signal terminal of the master single chip microcomputer, the second terminal of the first control device is of grounding; the first terminal of the relay coil connects to the cathode of the diode, the first terminal of the relay coil also connects to the first power supply, the second terminal of the relay coil connects to the anode of the diode, the second terminal of the relay coil also connects to the collector electrode of the triode, the base electrode of the triode connects to the first terminal of the first resistor, the second terminal of the first resistor connects to the master single chip microcomputer, the first terminal of the first resistor also connects to the first terminal of the second resistor, the second terminal of the second resistor connects to the emitting electrode of the triode, the emitting electrode of the triode is of grounding, the voltage input terminal of the second control chip connects to the first power supply, the voltage output terminal of the second control chip connects to the voltage input terminal of the master single chip microcomputer, the voltage output terminal of the second control chip connects to the first terminal of the thirteenth resistor and the first terminal of the ninth capacitor respectively, the second terminal of the thirteenth resistor connectors to the anode of the first light emitting diode, the cathode of the first light emitting diode connects to the control terminal of the first indicator of the master single chip microcomputer, the first terminal of the ninth capacitor also connects to the first terminal of the fourteenth resistor, the second terminal of the fourteenth resistor connects to the second power supply, the second terminal of the ninth resistor is of grounding, the grounding terminal of the second control chip and the grounding terminal of the master single chip microcomputer are of grounding, the first terminal of the eighth capacitor connects to the first power supply, the second terminal of the eighth capacitor is of grounding, the first interface and the second interface of the first signal interface are of grounding, the third interface and the fourth interface of the first signal interface connects to the signal terminal of the second indicator of the master single chip microcomputer, the fifth interface and the sixth interface of the first signal interface connects to the second light turning-on signal terminal of the master single chip microcomputer, the seventh interface of the first signal interface connects to the second power supply, the eighth interface of the first signal interface connects to the third light turning-on signal terminal of the master single chip microcomputer, the first terminal of the first switch connects to the signal terminal of the first clock of the master single chip microcomputer, the first terminal of the second switch connects to the signal terminal of the second clock of the master single chip microcomputer, the first terminal of the third switch connects to the signal terminal of the third clock of the master single chip microcomputer, the first terminal of the fourth switch connects to the signal terminal of the fourth clock of the master single chip microcomputer, the second terminal of the first-fourth switches is of grounding.

In addition, to achieve the aforesaid objective, the present invention also provides a man-free sterilization system control method which is applied to the aforesaid man-free sterilization system control device which comprises: a switch control module, a system control module, the first control circuit, the second control circuit and the third control circuit;

the man-free serialization system control method is as follows:

the first control circuit is designed for accessing the first light turning-on signal when there is not an operator in the space to be sterilized and sending the first light turning-on signal to the system control module;

the second control circuit is designed for accessing the second light turning-on signal when the door is closed in the space to be sterilized and sending the second light turning-on signal to the system control module;

the third control circuit is designed to access the third light turning-on signal after the operator leaves the space to be sterilized and closes the door and send the third light turning-on signal to the system control module;

the system control module is used for receiving the first light turning-on signal, the second light turning-on signal and the third light turning-on signal, when the time of receiving the first light turning-on signal is earlier than that of the third light turning-on signal, turning-on drive signal is generated according to the first light turning-on signal, the second light turning-on signal and the third light turning-on signal, and the turning-on drive signal is sent to the switch control module;

the switch control module is used for receiving the turning-on drive signal and controlling the tuning-on of the UV light according to the turning-on drive signal;

the second control circuit is also used for accessing the turning-off signal when the door is opened and sending the light turning-off signal to the system control module;

the system control module is also used for receiving the light turning-off signal, generating turning-off drive signals according to the light turning-off signal and sending the turning-off drive signal to the switch control module;

the switch control module is used for receiving the turning-off drive signal and controlling the tuning-off of the UV light according to the turning-off drive signal.

The present invention provides a man-free sterilization system control device which comprises: a switch control module, a system control module, the first control circuit, the second control circuit and the third control circuit; the signal input terminal of the system control module connects to the signal output terminal of the first control circuit, the signal output terminal of the second control circuit and the signal output terminal of the third control circuit, the the driving signal output terminal of the system control module connects to the driving signal input terminal of the switch control module, the switch control module is used for connecting the power and the UV light, the first control circuit is provided in the space to be sterilized, the second control circuit is provided at the door of the space to be sterilized, and the third control circuit is provided in the space to be sterilized; With the specific turning-on mode and the automatic turning-off function, the UV light is turned on for sterilization only when there are not people in the space to be sterilized. The man-free sterilization system control device can convert the indoor space into a sealed space for working of UV lights to improve the safety of UV lights and make UV lights with a low safety level be directly applied in a sealed indoor environment. UV lights can be used in an open space, which enables UV lights to sterilize both air in the space and the surface of irradiated objects and improves the sterilization effect of the device. The fan-free sterilization device can prevent the configuration of a sterilization cavity on the UV light, which can reduce the cost of the device. The man-free sterilization system control device can reduce the energy consumption and avoid noises. UV lights can be adjusted at will, which enables more flexible use of the device. In addition, light can be distributed freely, which can significantly improve the extension of the device and the use ratio of light emitted from the UV light source in the UV light and improve the sterilization efficiency.

### BRIEF DESCRIPTION OF THE DRAWINGS

FIG. 1 is a schematic diagram of Embodiment I of the man-free sterilization system control device of the present invention;

FIG. 2 is a installation diagram of Embodiment I of the man-free sterilization system control device of the present invention;

FIG. 3 is the exploded drawing of the first control module of the present invention;

FIG. 4 is the exploded drawing of the second control module of the present invention;

FIG. 5 is the first view of the first control module of the present invention;

FIG. 6 is the second view of the first control module of the present invention;

FIG. 7 is the view of the second control module of the present invention;

FIG. 8 is the display image of the display screen of the present invention;

FIG. 9 is the schematic diagram showing the circuit of the second control module of the present invention;

FIG. 10 is the schematic diagram showing the UV light control circuit of the man-free sterilization system control circuit of the present invention;

FIG. 11 is the schematic diagram showing the control switch of the man-free sterilization system control circuit of the present invention;

FIG. 12 is the flow chart of the Embodiment I of the control method of the man-free sterilization system of the present invention.

The implementation, functional characteristics and advantages of the present invention will be further illustrated hereinafter in conjunction with the embodiments and drawings.

### DETAILED DESCRIPTION

It should be understood, wherein the specific embodiment described is just used to explain the invention, not intended to limit the invention.

In the drawings, 10 represents the switch control module, 20 represents the system control module, 30 represents the first control circuit, 40 represents the second control circuit, 50 represents the third control circuit, F1 represents the fuse tube, VCC1˜VCC2 represents the first-second power supply, CON1 represents the power interface, CON2 represents the switch interface, U1˜U2 represents the first-second control chips, MCU represents the master single chip microcomputer, VDR1 represents the voltage-dependent resistor, DB1 represents the rectifier bridge, S1˜S3 represents the first-third control devices, RLY1 represents the relay armature, RLY2 represents the relay coil, LED1˜LED2 represents the first-second light emitting diode, R1˜R15 represents the first-fifteenth resistors, D1˜D4 represents the first-fourth diodes, Q1 represents the triode, J1˜J2 represents the first-second signal interfaces, C1˜C9 represents the first-ninth capacitors, L1˜L4 represents the first-fourth inductors, A represents the top shell, B represents the bottom shell, C represents the wiring hole, D represents the first wiring port, E represents the first extension interface, F represents the support frame, G represents the on-off key, H represents the single chip microcomputer and relay, I represents the indicator, J represents the key, K represents the key switch, L represents the display screen, M represents the second wiring port, N represents the second extension interface, 50 represents the third control circuit, F1 represents the fuse tube, VCC1˜VCC2 represents the first-second power, CON1 represents the power interface, CON2 represents the switch interface, U1˜U2 represents the first-second control chips, CU represents the master single chip microcomputer, VDR1 represents the voltage-dependent resistor, DB1 represents the rectifier bridge, S1˜S3 represents the first-third control devices, RLY1 represents the relay armature, RLY2 represents the relay coil, LED1˜LED2 represents the first-second diodes, R1˜R15 represents the first-fifteenth resistors, D1˜D4 represents the first-fourth diodes, Q1 represents the triodes, J1˜J2 represents the first-second signal interfaces, C1˜C9 represents the first-ninth capacitors, L1˜L4 represents the first-fourth inductors, A represents the top shell, B represents the bottom shell, C represents the wiring hole, D represents the first wiring port, E represents the first extension interface, F represents the support frame, G represents the switch key, H represents the single chip microcomputer and relay, I represents the indicator, J represents the key, K represents the key switch, L represents the display screen, M represents the second wiring port, N represents the second extension interface.

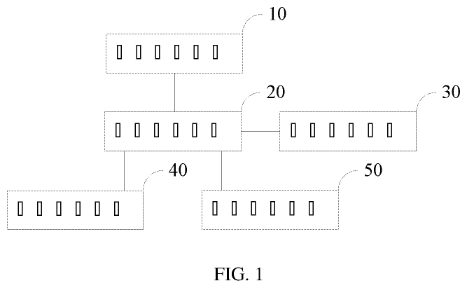

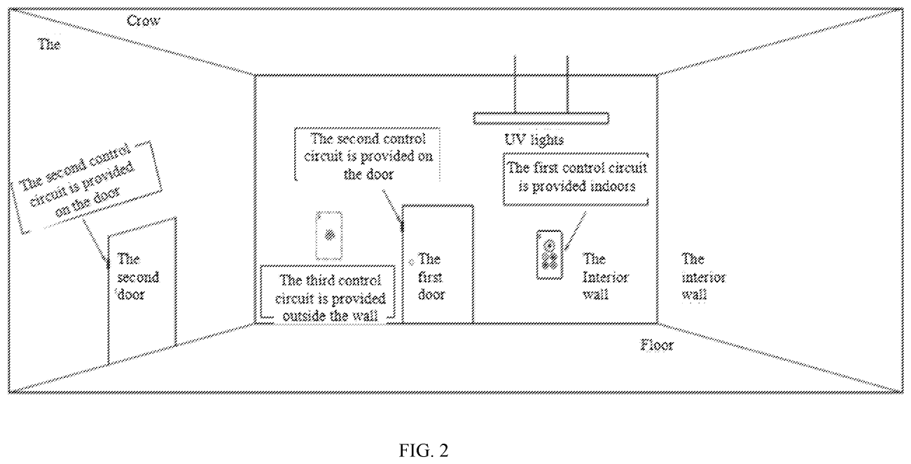

This embodiment of the present invention provides a man-free sterilization system control device (see FIG. 1 and FIG. 2). FIG. 1 is the schematic diagram of Embodiment I of the man-free sterilization system control device of the present invention, FIG. 2 is the drawing showing the installation of Embodiment I of the man-free sterilization system control device of the present invention, the man-free sterilization system control device comprises: a switch control module, a system control module, the first control circuit, the second control circuit and the third control circuit; the signal input terminal of the system control module connects to the signal output terminal of the first control circuit, the signal output terminal of the second control circuit and the signal output terminal of the third control circuit, the the driving signal output terminal of the system control module connects to the driving signal input terminal of the switch control module, the switch control module is used for connecting the power and the UV light, the first control circuit is provided in the space to be sterilized, the second control circuit is provided at the door of the space to be sterilized, and the third control circuit is provided in the space to be sterilized.

It should be noted that the connection mode of the first control circuit, the second control circuit and the third control circuit can be physical connection or wireless connection. The first control circuit can be provided on the inner wall of the space to be sterilized, the second control circuit can be provided on the door for accessing the space to be sterilized, and the third control circuit can be provided on the outer wall of the space to be sterilized, for which there is not a restriction in the present invention.

Understandably, the switch control module can connect to the AC power supply, that is the firing line and the null line of the AC power supply connect to the switch control module, and the switch control module can directly connect to the DC power supply, that is the cathode and the anode of the DC power supply connect to the switch control module. The switch control module connects to a UV light, or connects to a device with the sterilization function, for which there is not a restriction in the present invention.

Understandably, the space to be sterilized can be provided with one or several UV lights which can be installed in any position of the space to be sterilized at any angle and any altitude to sterilize the space to be sterilized, and the UV light can be used in the way where ordinary illuminating lights are used, for which there is not a restriction in the present invention.

The first control circuit is designed for accessing the first light turning-on signal when there is not an operator in the space to be sterilized and sending the first light turning-on signal to the system control module;

in the concrete execution, the first control circuit can be provided on the inner wall of the space to be sterilized, when the operator confirms upon inspection that there are not non-operation personnel in the space to be sterilized, the operator can press the switch command button on the inner wall of the space to be sterilized, so that the first control circuit will receive the first light turning-on signal and the first control circuit will send the first light turning-on signal to the system control module, for which there is not a restriction in the present invention.

The second control circuit is designed for accessing the second light turning-on signal when the door is closed in the space to be sterilized and sending the second light turning-on signal to the system control module;

in the concrete execution, the second control circuit can be provided on the door of the space to be sterilized, the door of the space to be sterilized can be a sliding door, when the operator leaves the space to be sterilized and closes its door, the second control circuit will receive the second light turning-on signal automatically and will send the second light turning-on signal to the system control module, for which there is not a restriction in the present invention.

The third control circuit is designed to access the third light turning-on signal after the operator leaves the space to be sterilized and closes the door and send the third light turning-on signal to the system control module;

in the concrete execution, the third control circuit can be provided on the outer wall of the space to be sterilized, when the operator leaves the space to be sterilized and closes its door, the operator can insert the key into the lock hole on the outer wall of the space to be sterilized, after the operator rotates the key, the third control circuit will receive the third light turning-on signal and send the third light turning-on signal to the system control module, for which there is not a restriction in the present invention;

in the concrete execution, the third control circuit can be provided on the outer wall of the space to be sterilized, when the operator leaves the space to be sterilized and closes its door, the operator can insert the key into the lock hole on the outer wall of the space to be sterilized, after the operator rotates the key, the third control circuit will receive the third light turning-on signal and send the third light turning-on signal to the system control module, for which there is not a restriction in the present invention.

The system control module is used for receiving the first light turning-on signal, the second light turning-on signal and the third light turning-on signal, when the time of receiving the first light turning-on signal is earlier than that of the third light turning-on signal, turning-on drive signal is generated according to the first light turning-on signal, the second light turning-on signal and the third light turning-on signal, and the turning-on drive signal is sent to the switch control module;

Understandably, the system control module can receive the first light turning-on signal, the second light turning-on signal and the third light turning-on signal in the way of physical connection or wireless connection. In the meanwhile, the system control module will generate the turning-on drive signal according to the first light turning-on signal, the second light turning-on signal and the third light turning-on signal and send the turning-on drive signal to the switch control module only when the time of receiving the first light turning-on signal is earlier than the time of receiving the third light turning-on signal. When the condition cannot be met, the system control module cannot generate the turning-on drive signal.

The switch control module is used for receiving the turning-on drive signal and controlling the tuning-on of the UV light according to the turning-on drive signal;

understandably, the switch control module can be a device with the switch function, when the switch control module receives the turning-on drive signal, the switch control module's switch can be turned off, the switch control module provides working voltage for UV lights based on the drive signal to make the UV light turn on to sterilize the space to be sterilized.

The second control circuit is also used for accessing the turning-off signal when the door is opened and sending the light turning-off signal to the system control module;

understandably, the space to be sterilized can have one door or several doors, when any door is opened, the second control circuit can automatically receive the light turning-off signal, and the second control circuit will send the light turning-off signal to the system control module.

The system control module is also used for receiving the light turning-off signal, generating turning-off drive signals according to the light turning-off signal and sending the turning-off drive signal to the switch control module;

understandably, whenever the light turning-off signal is received, the system control module will generate turning-off drive signal immediately and will send the turning-off drive signal to the switch control module.

The switch control module is used for receiving the turning-off drive signal and controlling the tuning-off of the UV light according to the turning-off drive signal;

understandably, when the switch control module receives the turning-off drive signal, the switch control module's switch can be turned off, the switch control module provides working voltage for UV lights based on the drive signal to make the UV light turn on to sterilize the space to be sterilized.

In the embodiment, the signal input terminal of the system control module connects to the signal output terminal of the first control circuit, the signal output signal of the second control circuit and the signal output terminal of the third control circuit respectively, the drive signal output terminal of the system control module connects to the drive signal input terminal of the switch control module, the switch control module connects to the first power supply and the UV light respectively, the first control circuit is provided in the space to be sterilized, the second control circuit is provided at the door of the space to be sterilized and the third control circuit is provided outside the space to be sterilized. the man-free sterilization system control device judges whether there are people in the room in the order of operation of three control devices set indoors, at the door and outdoors, the UV light is turned on when it is confirmed that there is no people in the room, the device will turn off the UV light man-freely when it is judged that there may be people in the room. With the specific turning-on mode and the automatic turning-off function, the UV light is turned on for sterilization only when there are not people in the space to be sterilized. The man-free sterilization system control device can convert the indoor space into a sealed space for working of UV lights to improve the safety of UV lights and make UV lights with a low safety level be directly applied in a sealed indoor environment. UV lights can be used in an open space, which enables UV lights to sterilize both air in the space and the surface of irradiated objects and improves the sterilization effect of the device. The fan-free sterilization device can prevent the configuration of a sterilization cavity on the UV light, which can reduce the cost of the device. The man-free sterilization system control device can reduce the energy consumption and avoid noises. UV lights can be adjusted at will, which enables more flexible use of the device. In addition, light can be distributed freely, which can significantly improve the extension of the device and the use ratio of light emitted from the UV light source in the UV light and improve the sterilization efficiency.

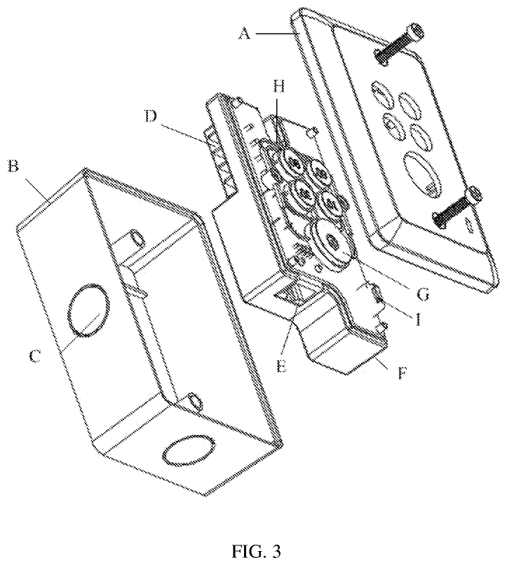

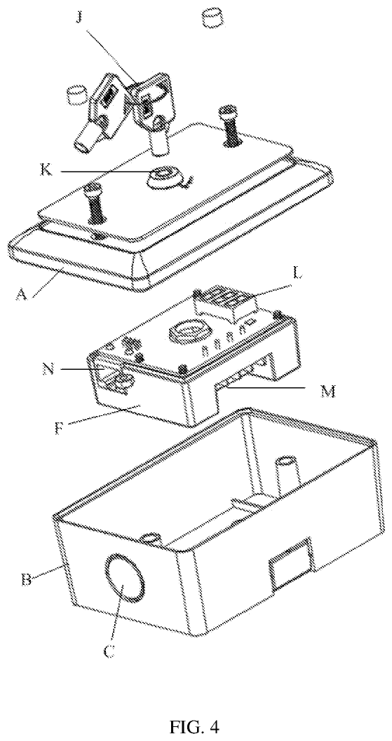

Please refer to FIG. 3 and FIG. 4. FIG. 3 is the exploded drawing of the first control module of the present invention, and FIG. 4 is the exploded drawing of the second control module of the present invention.

The switch control module, the system control module and the first control circuit form the independent first control module;

and/or, the third control circuit is an independent second control module.

Understandably, the switch control module, the system control module and the first control circuit can be combined into the independent first control module, and the third control circuit can be the independent second control module to simplify the circuit, make control operation simplifier and realize the sterilization function without operation of professionals.

Understandably, the shell of the first control module includes the top shell and the bottom shell, the shell can be provided with several wiring holes in any shape, the inside of the shell is provided with a support, the support is provided with an indicator and an on-off key, the first wiring port and the first extension interface. The on-off key can be used to provide the first light turning-on signal or the on-off signal, and the indicator can be used to remind the operator that the signal has been sent when the indicator goes on. The first wiring port can be used to connect the power supply, when the power supply is AC power, the first wiring port can connect to the firing line and the null line, and when the power supply is DC current, it can connect to the cathode and the anode. The first extension interface can be extended based on the actual need of the operator, for which there is not a restriction in the embodiment.

Understandably, the shell of the second control module includes the top shell and the bottom shell, the shell can be provided with several wiring holes in any shape, the top shell is provided with a key switch, the inside of the shell is provided with a support, the support is provided with a displayer, the second wiring port and the second extension interface, the key switch can be a normally closed switch with a spring-back effect where the key will spring back automatically after being rotated. the display screen is used for displaying the work time or the work state of the UV light. The second wiring port can be used to connect to the signal line of the magnetic door contact, the second wiring port can also connect to the first extension interface to send the door closing signal to the single chip microcomputer. The first extension interface can be extended based on the actual need of the operator, for which there is not a restriction in the embodiment.

The switch control module is a relay;

and/or, the system control module is a single chip microcomputer control circuit;

and/or, the second control circuit is a magnetic door contact circuit device or a mechanical switch circuit, the door automatically accesses the second light turning-on signal when the door is closed, and the door automatically accesses the light turning-off signal when the door is opened.

Understandably, the switch control module is a relay which is used to realize the switch control function, when the relay is powered on, the switch of the switch control module can be on, so that the UV light will turn on, and when the relay is powered off, the switch of the switch control module can be off to make the UV light turn off.

Understandably, the system control module can be a single chip microcomputer control circuit which is used to realize the intelligent control of the sterilization system control device, make the control operation safer and realize the sterilization function without operation of professionals.

Understandably, the second control circuit can be a magnetic door contact circuit or a mechanical switch circuit as needed, for which there is not a restriction in this embodiment.

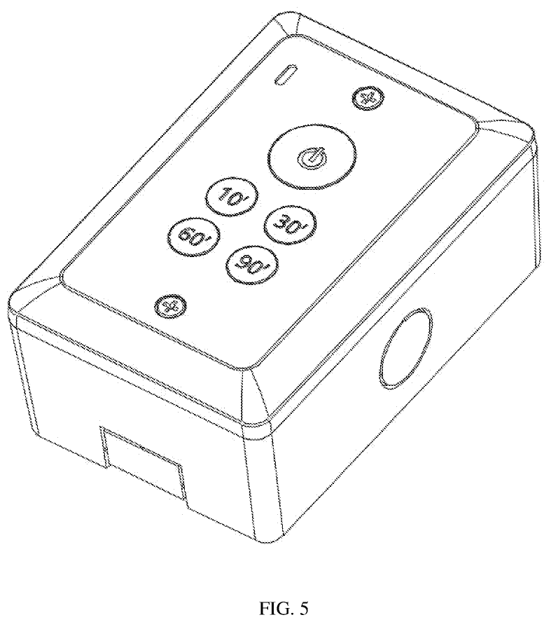

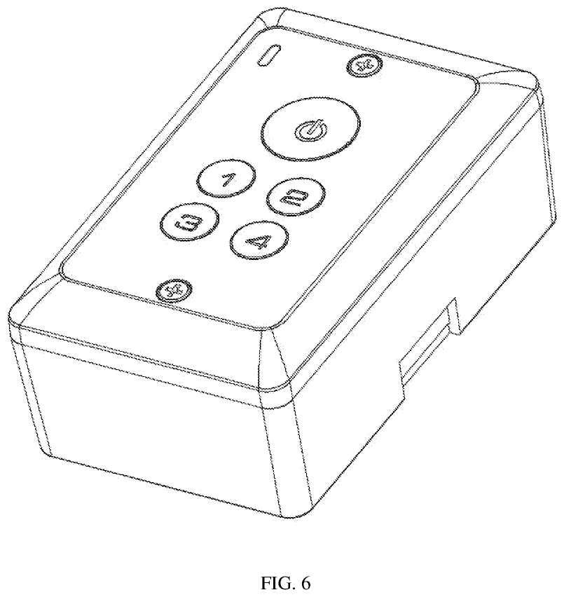

Please refer to FIG. 5 and FIG. 6. FIG. 5 is the first view of the first control module of the present invention and FIG. 6 is the second view of the first control module of the present invention.

The first control circuit comprises one or several light turning-on key/s, the light turning-on key/s provide/s the first light turning-on signal;

when there is only one light turning-on key, this light turning-on key is used for providing the first light turning-on signal or turning-off signal;

when there are several light turning-on keys, one of these light turning-on keys is used for providing the first light turning-on signal or turning-off signal, and other light turning-on keys are used for setting the light turning-on time.

In the concrete execution, the first control circuit can be provided on the inner wall of the space to be sterilized, when the operator confirms upon inspection that there are not non-operation personnel in the space to be sterilized, the operator can press the switch command button on the inner wall of the space to be sterilized, so that the first control circuit will receive the first light turning-on signal and the first control circuit will send the first light turning-on signal to the system control module, the operator can also press the time setting button on the inner wall of the space to be sterilized to make the UV light conducts sterilization within a specified time and goes off after the time is up to optimize the control function of the sterilization system control device, for which there is not a restriction in the present invention.

In the concrete execution, the first control module can be provided with a light turning-on key which can provide the first light turning-on signal or on-off signal, this light turning-on key can be displayed with the available power symbol, and the light turning-on key used to set the light turning-on time can be displayed with the corresponding preset time digits and can also be numbered in the time sequence from small to large or from large to small, for which there is not a restriction in the present invention.

The third control circuit comprises one or several light turning-on key/s, the light turning-on key/s provide/s the third light turning-on signal;

when there is only one light turning-on key, this light turning-on key is used for providing the third light turning-on signal or turning-off signal;

when there are several light turning-on keys, one of these light turning-on keys is used for providing the third light turning-on signal or turning-off signal, and other light turning-on keys are used for setting the light turning-on time.

In the concrete execution, the third control circuit can be provided on the outer wall of the space to be sterilized, when the operator leaves the space to be sterilized and closes its door, the operator can insert the key into the lock hole on the outer wall of the space to be sterilized, after the operator rotates the key, the third control circuit will receive the third light turning-on signal and send the third light turning-on signal to the system control module, for which there is not a restriction in the present invention.

In the concrete execution, the third control circuit can be provided on the outer wall of the space to be sterilized, when the operator leaves the space to be sterilized and closes its door, the operator can insert the key into the lock hole on the outer wall of the space to be sterilized, after the operator rotates the key, the third control circuit will receive the third light turning-on signal and send the third light turning-on signal to the system control module, for which there is not a restriction in the present invention.

The first control circuit comprises the first indicator;

the first indicator is used for reminding the operator of that the first light turning-on signal has been sent when the first indicator goes on;

and/or, the third control circuit comprises the second indicator which is used for reminding the operator of that the third light turning-on signal has been sent when the second indicator goes on.

Understandably, when the first indicator goes on, it reminds the operator that the first light turning-on signal has been sent to the system control module, if the first indicators fails to turn on, it reminds the operator that the operation is unsuccessful or the device breaks down, so that the operator can re-operate the device or inspect the device. When the second indicator goes on, it reminds the operator that the second light turning-on signal has been sent to the system control module, if the second indicators fails to turn on, it reminds the operator that the operation is unsuccessful or the device breaks down, so that the operator can re-operate the device or inspect the device.

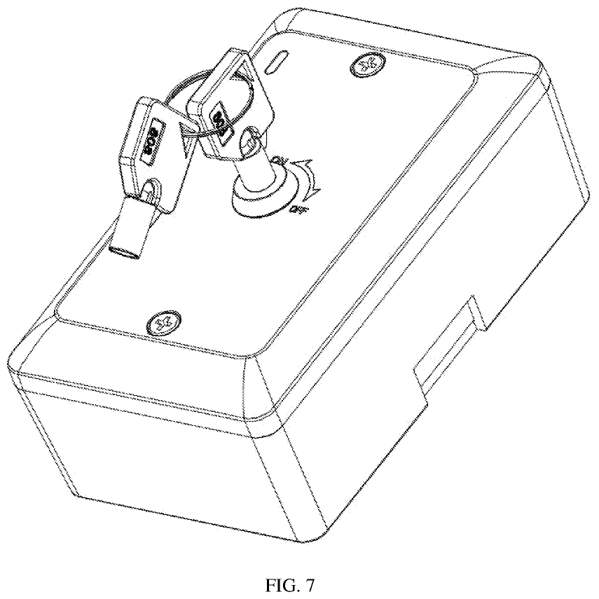

Please refer to FIG. 7 which is the display image of the second control module of the present invention.

The third control circuit comprises the enabling unit which enables the preset tool to make the third control circuit work normally.

The enabling unit is a key switch, and the preset tool is a specific key.

Understandably, when the enabling unit is enabled by the preset tool, normal work of the third control circuit can be controlled, and when the enabling unit is not enabled, the third control circuit is under the non-working state. The enabling unit can be a key switch, and the preset tool can be a special key or screwdriver, for which there is not a restriction in this embodiment.

Understandably, the enabling unit can also be a lock controlling the use of the third control circuit, preventing other non-operation personnel enabling the UV light.

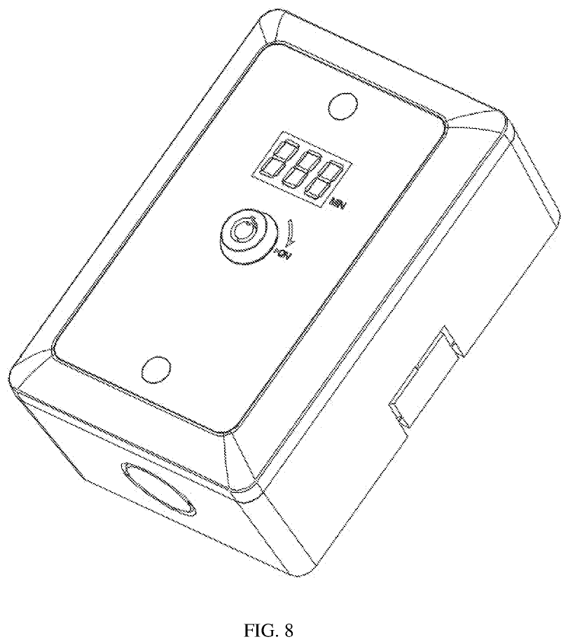

Please refer to FIG. 8 which is the display image of the display screen of the present invention.

The third control circuit comprises a display screen;

the display screen is used for displaying the work time or the work state of the UV light.

Understandably, the display screen can display the working condition of the UV light, the working condition can be the generated working duration or the preset working duration of the UV light, for which there is not a restriction in this embodiment.

In the concrete execution, the operator can set the preset working duration as 30 min. If it has been 10 min since the UV light was turned on, the 30 min preset working duration in red or 10 min of generated working duration in green can be displayed on the display screen, the display screen can also display the working time and condition in a cyclical way based on a preset cycle, the preset cycle and the display color can be set according to the actual need of the operator, for which there is not a restriction in this embodiment.

The system control module comprises the first extension interface unit, the third control circuit comprises the second extension interface unit, the first extension interface unit and the second extension interface unit are multi-way interfaces, the control signal terminal of the second control circuit connects to the control signal terminal of the second extension interface unit, and the first extension interface unit connects to the second extension interface unit via the control signal cable.

Understandably, the control signal cable can transmit the control signal of the second control circuit and the third control circuit to the system control module to simplify circuit arrangements.

Understandably, the first extension interface unit connects to the second extension interface unit in a wireless way, the second control circuit and the third control circuit can be provided with wireless control signal transmitting device respectively, the system control module can be provided with corresponding wireless control signal receiving device to receive control signals of the second control circuit and the third control circuit so as to control turning-on and turning-off of the UV light.

The second control circuit comprises several control signal launch units which are provided in different door positions in a serial connection way;

the several control signal launch units are used for sending the second light turning-on signal to the system control module when doors corresponding to all the control signal launch units are closed;

the several control signal launch units are used for sending the light turning-off signal to the system control module when doors corresponding to any control signal launch units are open;

Understandably, when the space to be sterilized has several doors, each door should be provided with the control device of the second control circuit, several control devices should be connected in a serial connection way and then they are introduced into the system control module to ensure that the UV light is turned on only when all the doors are closed, and when any door is opened, the UV light can be turned off automatically; when the space to be sterilized has windows, if the UV rays can be accessed outside the window, windows should also be provided with the control device of the second control circuit to remind the operator of closing windows when the UV light is turned on.

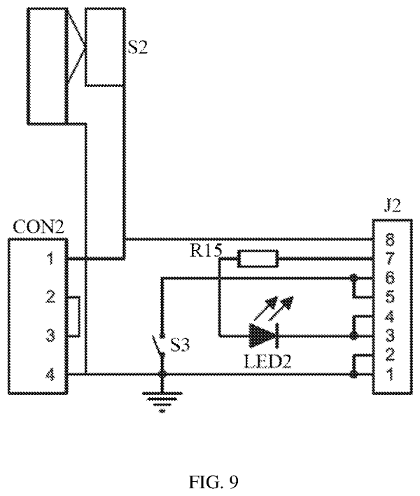

Please refer to FIG. 9 which is the schematic diagram showing the circuit of the second control module of the present invention.

In this embodiment, the second control module includes: the second control device, the third control device, the second signal interface, switch interface, the fifteenth resistor and the second light emitting diode;

the first terminal of the second control device connects to the first interface of the switch interface, the second terminal of the second control device connects to the fourth interface of the switch interface, the second interface of the switch interface connects to the third interface, the first interface of the switch interface also connects to the eighth interface of the second signal interface, the fourth interface of the switch interface also connects to the first interface of the second signal interface, the fifth interface and the sixth interface of the second signal interface also connect to the first terminal of the third control device, the second terminal of the third control devices is of grounding, the third interface and the fourth interface of the second signal interface connect to the cathode of the second light emitting diode, the anode of the second light emitting diode connects to the first terminal of the fifteenth resistor, the second terminal of the fifteenth resistor connects to the seventh interface of the second signal interface.

Understandably, the fifteenth resistor is a load resistor which is used to maintain normal work of the second light emitting diode.

Understandably, the second control device can be a magnetic door contact or any device installed onto the door, when the door is closed, the second control device is disenabled to make the switch interface receive the second light turning-on signal which is sent by the second signal interface to the first signal interface and then sent by the first signal interface to the master single chip microcomputer.

Understandably, when the space to be sterilized has several doors, each door can be provided with the second control device, and several switches connect switch interfaces in a serial connection way to realize that the master single chip microcomputer receives the second switch signal only when all the doors are closed.

Understandably, the third control device can connect to the lock hole or switch command button provided on the outer wall of the space to be sterilized, when the lock hole is rotated with a key or the switch command button is pressed, the third control device is switched on, so that the second signal interface receives the third light turning-on signal which is sent by the second signal interface to the first signal interface and then sent by the first signal interface to the master single chip microcomputer.

Understandably, the control equipment and the third control device can also be integrated through wiring and share one cable to transmit signals to the master single chip microcomputer, simplifying wire arrangement of the man-free sterilization system control device.

Understandably, the second signal interface sends the third light turning-on signal to the first signal interface which sends the third light turning-on signal to the master single chip microcomputer, when the master single chip microcomputer receives the third control command, the master single chip microcomputer can control the second light emitting diode to be powered on to make the second light emitting diode illuminate, so that it can remind the operator that the man-free sterilization system control device has received the third light turning-on signal and the UV light has begun to work.

The switch control module, the system control module, the first control circuit and the first control signal extension port, the signal input terminal of the system control module connects to the signal output terminal of the first control circuit and the signal output terminal of the first control signal extension port, the drive signal output terminal of the system control module connects to the drive signal input terminal of the switch control module, and the switch control module connects to the power supply and the UV light;

the first control circuit is designed for accessing the first light turning-on signal when there is not an operator in the space to be sterilized and sending the first light turning-on signal to the system control module;

the first control signal extension port is used for accessing the second light turning-on signal when the door of the space to be sterilized is closed and sending the second light turning-on signal to the system control module;

the first control signal extension port is designed to access the third light turning-on signal after the operator leaves the space to be sterilized and closes the door and send the third light turning-on signal to the system control module;

the system control module is used for receiving the first light turning-on signal, the second light turning-on signal and the third light turning-on signal, when the time of receiving the first light turning-on signal is earlier than that of the third light turning-on signal, turning-on drive signal is generated according to the first light turning-on signal, the second light turning-on signal and the third light turning-on signal, and the turning-on drive signal is sent to the switch control module;

the switch control module is used for receiving the turning-on drive signal and controlling the tuning-on of the UV light according to the turning-on drive signal;

the first control signal extension port is also used for accessing the turning-off signal when the door is opened and sending the light turning-off signal to the system control module;

the system control module is also used for receiving the light turning-off signal, generating turning-off drive signals according to the light turning-off signal and sending the turning-off drive signal to the switch control module;

the switch control module is used for receiving the turning-off drive signal and controlling the tuning-off of the UV light according to the turning-off drive signal;

Understandably, the switch control module can connect to the AC power supply, that is the firing line and the null line of the AC power supply connect to the switch control module, and the switch control module can directly connect to the DC power supply, that is the cathode and the anode of the DC power supply connect to the switch control module.

Understandably, the first control signal extension port connects to the system control module electrically, when the first control signal extension port provides the system control module with one another light turning-on signal or several other light turning-on signals, the system control module switches on the control switch based on the light turning-on signal to make the UV light illuminate. When the first control signal extension port provides one or several light turning-off signals for the system control module, the system control module will switch off the control switch based on the light turning-off signal and then the UV light goes off.

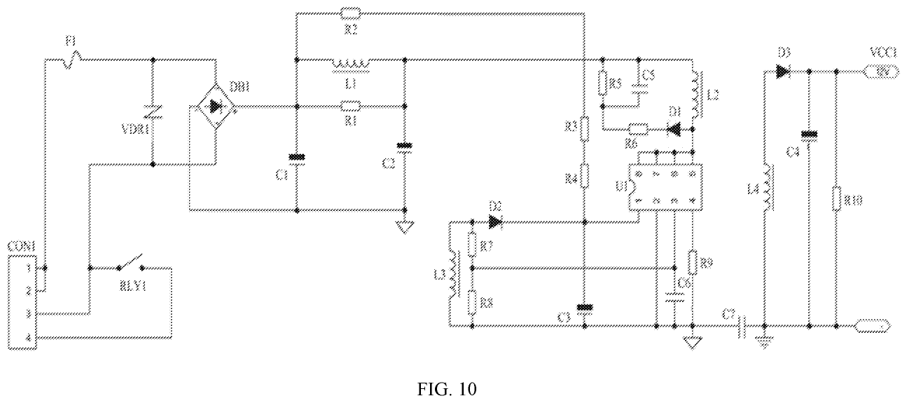

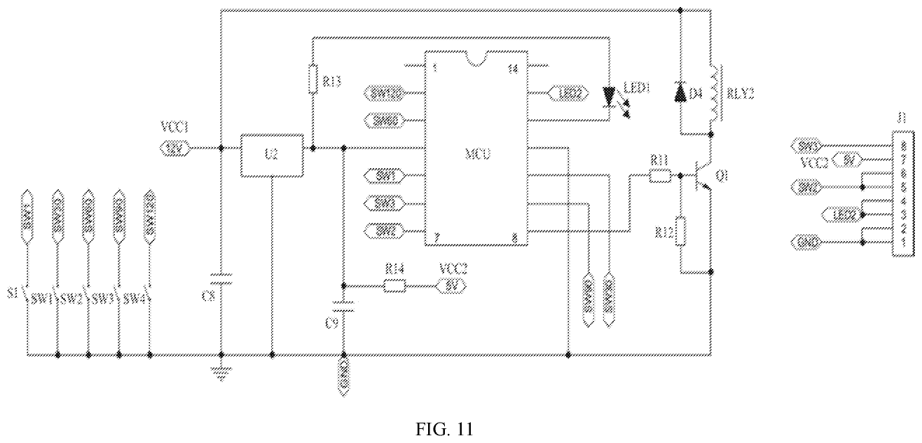

Please refer to FIG. 10 and FIG. 11. FIG. 10 is the schematic diagram showing the UV light control circuit of the man-free sterilization system control circuit of the present invention, and FIG. 11 is the schematic diagram showing the control switch circuit of the man-free sterilization system control circuit of the present invention.

In this embodiment, the man-free sterilization system control device provided by the present invention includes: power interfaces, a fuse tube, a voltage-dependent resistor, a rectifier bridge, a relay armature, a relay coil, the first power supply-the second power supply, the first control chip-the second control chip, the first control device, a master single chip microcomputer, the first diode-the fourth diode, a triode, the first resistor-the fourteen resistor, the first signal interface, the first capacitor-the fourth capacitor, the first capacitor-the ninth capacitor, the first inductor-the fourth inductor, the first switch-the fourth switch and the first light emitting diode;

the first interface and the second interface of the power interface connects to the first terminal of the fuse tube, the second terminal of the fuse tube connects to the first terminal of the voltage-dependent resistor and the first AC input terminal of the rectifier bridge, the third interface of the power interface connects to the voltage input terminal of the step-down rectifier unit, the third interface of the power interface connects to the second terminal of the voltage-dependent resistor and the second AC input terminal of the rectifier bridge respectively, the fourth interface of the power interface connects to the second terminal of the relay armature, the positive output terminal of the rectifier bridge connects to the first resistor and the second resistor respectively, the positive output terminal of the rectifier bridge also connects to the first terminal of the first inductor and the first terminal of the first capacitor respectively, the negative output terminal of the rectifier bridge is of grounding, the first terminal of the second capacitor connects to the second terminal of the first resistor and the second terminal of the first inductor respectively, the second terminal of the first capacitor and the second terminal of the second capacitor are of grounding, the second terminal of the first inductor connects to the first terminal of the fifth capacitor and the first terminal of the fifth resistor respectively, the first terminal of the sixth resistor connects to the second terminal of the fifth capacitor and the second terminal of the fifth resistor respectively, the second terminal of the sixth resistor connects to the cathode of the first diode, the second terminal of the first inductor connects to the first terminal of the second inductor, the fifth-eighth interfaces of the first control chip connect to the anode of the first diode and the second terminal of the second inductor, the second terminal of the second resistor connects to the first terminal of the third resistor, the second terminal of the third resistor connects to the first terminal of the fourth resistor, the first interface of the first control chip connects to the cathode of the second diode and the first terminal of the third capacitor respectively, the second terminal of the fourth resistor connects to the first terminal of the third capacitor, the anode of the second diode connects to the first terminal of the third inductor and the first terminal of the seventh resistor respectively, the second terminal of the seventh resistor connects to the first terminal of the eighth resistor and the first terminal of the sixth capacitor, the second terminal of the third inductor and the second terminal of the eighth resistor are of grounding, the third interface of the first control chip connects to the first terminal of the sixth capacitor, the second terminal of the third capacitor and the second terminal of the sixth capacitor are of grounding, the second interface of the first control chip is of grounding, the fourth interface of the first control chip connects to the first terminal of the ninth resistor, the second terminal of the ninth resistor connects to the first terminal of the seventh capacitor, the second terminal of the ninth resistor is of grounding, the second terminal of the seventh capacitor connects to the first terminal of the fourth inductor, the first terminal of the fourth inductor is of grounding, the second terminal of the fourth inductor connects to the anode of the third diode, the cathode of the third diode connects to the first terminal of the fourth capacitor and the first terminal of the tenth resistor respectively, the cathode of the third diode also connects to the first power supply, the second terminal of the fourth capacitor and the second terminal of the tenth resistor are of grounding.

Understandably, under the action of the electromagnetic attraction, the contact points of the relay armature will be closed, so that the UV light will get the 12V working voltage and go on, and the 12V voltage can also serve as the first power supply.

Understandably, the fuse tube can serve as an electrical component which guarantees safe operation of the circuit, the fuse tube will fuse when the current or temperature rises to a certain value abnormally, and then the current will be cut off to protect the safe operation of the circuit and the safety coefficient will be strengthened significantly.

Understandably, the rectifying action of the rectifier bridge is achieved through the one-way conducting of diodes, the rectifier bridge can convert AC current whose electrical level fluctuates around zero into DC current which can be normally used by UV lights and other electrical elements by virtue of the one-way conducting characteristic of diodes.

Understandably, voltage-dependent resistor is a voltage limiting type protective device, the control circuit can make use of the non-linear characteristic of the voltage-dependent resistor, when overvoltage happens to two electrodes of the voltage-dependent resistor, the voltage-dependent resistor can clamp voltage to a relatively fixed voltage value, when the voltage imposed on the voltage-dependent resistor is lower than its threshold value, the voltage-dependent resistor is equivalent to a switch under the off state, when the voltage imposed on the voltage-dependent resistor is higher than its threshold voltage, the voltage-dependent resistor is equivalent to a switch under the on state for protecting the control circuit.

Understandably, the first-tenth resistors are load resistors for protecting the safety of circuits to make circuit components work normally.

Understandably, the first-seventh capacitors can store partial energy and improve the system voltage to reduce energy loss and improve the stability of the system.

Understandably, the first-fourth capacitors are of power filtering, decoupling, signal coupling and DC resisting, and the electric capacity of the electrolytic capacitor is very large and the cost is relatively low, so it can reduce the cost when improving the stability of the control circuit.

Understandably, the first-fourth inductors are circular coil inductors, when there is current in the coil, a magnetic field around the coil will be formed, so that the circular coil inductor will be of filtering, oscillation and delaying, the control circuit will be of strong stability and higher safety.

The first terminal of the first control device connects to the first light turning-on signal terminal of the master single chip microcomputer, the second terminal of the first control device is of grounding; the first terminal of the relay coil connects to the cathode of the diode, the first terminal of the relay coil also connects to the first power supply, the second terminal of the relay coil connects to the anode of the diode, the second terminal of the relay coil also connects to the collector electrode of the triode, the base electrode of the triode connects to the first terminal of the first resistor, the second terminal of the first resistor connects to the master single chip microcomputer, the first terminal of the first resistor also connects to the first terminal of the second resistor, the second terminal of the second resistor connects to the emitting electrode of the triode, the emitting electrode of the triode is of grounding, the voltage input terminal of the second control chip connects to the first power supply, the voltage output terminal of the second control chip connects to the voltage input terminal of the master single chip microcomputer, the voltage output terminal of the second control chip connects to the first terminal of the thirteenth resistor and the first terminal of the ninth capacitor respectively, the second terminal of the thirteenth resistor connectors to the anode of the first light emitting diode, the cathode of the first light emitting diode connects to the control terminal of the first indicator of the master single chip microcomputer, the first terminal of the ninth capacitor also connects to the first terminal of the fourteenth resistor, the second terminal of the fourteenth resistor connects to the second power supply, the second terminal of the ninth resistor is of grounding, the grounding terminal of the second control chip and the grounding terminal of the master single chip microcomputer are of grounding, the first terminal of the eighth capacitor connects to the first power supply, the second terminal of the eighth capacitor is of grounding, the first interface and the second interface of the first signal interface are of grounding, the third interface and the fourth interface of the first signal interface connects to the signal terminal of the second indicator of the master single chip microcomputer, the fifth interface and the sixth interface of the first signal interface connects to the second light turning-on signal terminal of the master single chip microcomputer, the seventh interface of the first signal interface connects to the second power supply, the eighth interface of the first signal interface connects to the third light turning-on signal terminal of the master single chip microcomputer, the first terminal of the first switch connects to the signal terminal of the first clock of the master single chip microcomputer, the first terminal of the second switch connects to the signal terminal of the second clock of the master single chip microcomputer, the first terminal of the third switch connects to the signal terminal of the third clock of the master single chip microcomputer, the first terminal of the fourth switch connects to the signal terminal of the fourth clock of the master single chip microcomputer, the second terminal of the first-fourth switches is of grounding.

Understandably, the first control device can connect to the light turning-on indication button on the inner wall of the space to be sterilized, when the light turning-on indication button is pressed, the first control device will be switched on to make the master single chip microcomputer receive the first light turning-on signal.

Understandably, the first signal interface can receive the second light turning-on signal and the third light turning-on signal sent by the third control circuit and send the second light turning-on signal and the third light turning-on signal to the master single chip microcomputer to make the master single chip microcomputer generate drive signals based on the first light turning-on signal, the second light turning-on signal and the third light turning-on signal.

Understandably, the master single chip microcomputer increases the current of the base electrode of the triode based on the drive signal, when the current of the base electrode of the triode increases and the current of the collector electrode cannot be increased continuously, the triode goes into the saturated state, the voltage between the collector electrode and the transmitting electrode is small, and the triode in such a case can serve as a switch under the on state.

Understandably, the relay coil is a coil wound on an iron core, when there is current flowing inside the coil, the electromagnetic effect will be generated, the armature will get close to the iron core under the attraction action of the electromagnetic force, after the triode shows great current, there will be current flowing inside the coil of the relay, and the contact points of the relay armature will be closed.

Understandably, the fourth diode is a freewheel diode, coil can store energy when the coil is electrified, the coil will resist sudden change of current (i.e. electromagnetic induction), so that current can only get larger or smaller slowly, when the coil is losing power, large voltage will be generated between two ends of the coil, which may damage the coil, a loop between the fourth diode and the coil may be generated to run out of the stored energy.

Understandably, the eleventh-fourteenth resistors are load resistors, and the eleventh resistor is used to guarantee normal work of the triode. The twelfth resistor can guarantee reliable cut-off of the triode when no voltage is input into the triode or the input terminal suspends, the thirteenth resistor can protect normal work of the first light emitting diode, the fourteenth resistor featuring voltage step-down and rectifying can output 5V voltage which can serve as the second power supply.

Understandably, the eighth-ninth capacitors can store partial energy and improve the system voltage to reduce energy loss and improve the stability of the system.

Understandably, the first switch, the second switch, the third switch and the fourth switch are timing switches, the first switch, the second switch, the third switch and the fourth switch can connect to the timing button provided on the inner or outer wall of the space to be sterilized to turn off the UV light 30 min after working of the first switch, 60 min after working of the second switch, 90 min after working of the third switch and 120 min after working of the fourth switch, the preset light turning-off time can also be set by the operator as needed, for which there is not a restriction in the present invention. When the light turning-off time is up, the UV light will go off automatically.

Understandably, when the master single chip microcomputer receives the first control command, it can control illuminating of the first light emitting diode to remind the operator that the man-free sterilization system control device has received the first light turning-on signal.

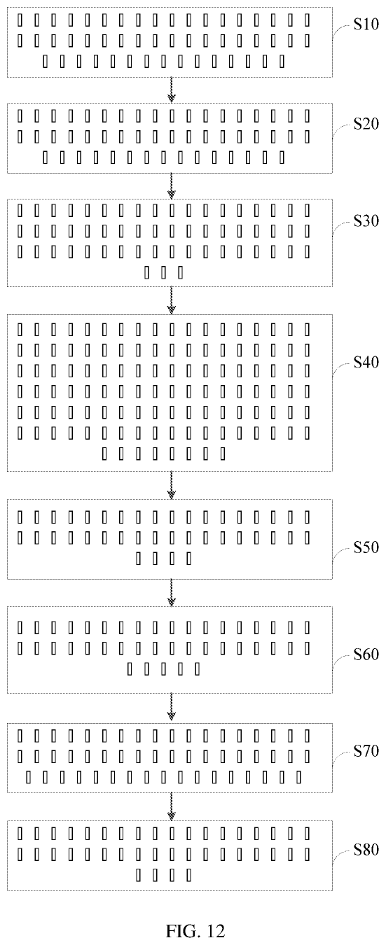

Furthermore, FIG. 12 is the flow chart of the Embodiment I of the control method of the man-free sterilization system of the present invention. the man-free serialization system control device is as follows: a switch control module, a system control module, the first control circuit, the second control circuit and the third control circuit.

The man-free serialization system control method is as follows:

S10: the first control circuit is designed for accessing the first light turning-on signal when there is not an operator in the space to be sterilized and sending the first light turning-on signal to the system control module;

S20: the second control circuit is designed for accessing the second light turning-on signal when the door is closed in the space to be sterilized and sending the second light turning-on signal to the system control module;

S30: the third control circuit is designed to access the third light turning-on signal after the operator leaves the space to be sterilized and closes the door and send the third light turning-on signal to the system control module;

S40: the system control module is used for receiving the first light turning-on signal, the second light turning-on signal and the third light turning-on signal, when the time of receiving the first light turning-on signal is earlier than that of the third light turning-on signal, turning-on drive signal is generated according to the first light turning-on signal, the second light turning-on signal and the third light turning-on signal, and the turning-on drive signal is sent to the switch control module;

S50: the switch control module is used for receiving the turning-on drive signal and controlling the tuning-on of the UV light according to the turning-on drive signal;

S60: the second control circuit is also used for accessing the turning-off signal when the door is opened and sending the light turning-off signal to the system control module;

S70: the system control module is also used for receiving the light turning-off signal, generating turning-off drive signals according to the light turning-off signal and sending the turning-off drive signal to the switch control module;

S80: the switch control module is used for receiving the turning-off drive signal and controlling the tuning-off of the UV light according to the turning-off drive signal.

Understandably, the man-free sterilization system control device can judge whether there are people in the space to be sterilized through the operation order of three control devices set indoors, on the door and outside the door, the UV light is turned on when it is confirmed that there are no people inside the room, the UV light will be turned off automatically when it is confirmed that there may be people in the room, realizing sterilization of the space to be sterilized by the UV light through intelligent control.

Moreover, the term “comprise”, “contain” or other variations is intended to cover non-exclusive inclusion so that a process, method, article or device covering a series of elements not only includes those elements, but also other elements not explicitly listed, or the elements inherent to such process, method, article or device. Therefore, in the case without more restrictions, the elements limited by the statement “comprise a . . . ” do not exclude the existence of other identical elements in the process, method, article or system.

The above embodiment numbers of the present invention are only set for descriptions and do not represent the ranking of embodiments.

Through descriptions about the above modes of execution, technicians in this field can clearly know that above embodiments can be realized with the help of software and necessary general hardware platforms. Of course, hardware can be used, but the former one is the best mode of execution in most cases. Based on such an understanding, it can be the that the part of the technical scheme of the present invention in essence or contributive to existing technology can be embodied in the form of software products. This computer software product is stored in a storage medium (e.g. ROM/RAM, disk and CD), including several commands, to make one set of terminal equipment (e.g. mobile phone, computer, server, AC or network equipment) execute the method as described in each embodiment of the present invention.

The above is just the preferred embodiments of the present invention, not intended to limit the protection scope of the present invention. All the equivalent structure or the equivalent flow transfer which is made using the present invention manual and the attached drawings, the direct or the indirect application in other relevant technology fields are similarly all covered in the protection scope of the present invention.

1-15. (canceled)

16. A man-free sterilization system control device comprising:
a switch control module, a system control module, a first control circuit, a second control circuit and a third control circuit; a signal input terminal of the system control module connects to a signal output terminal of the first control circuit, a signal output terminal of the second control circuit and a signal output terminal of the third control circuit, a driving signal output terminal of the system control module connects to a driving signal input terminal of the switch control module, the switch control module is used for connecting power and a UV light, the first control circuit is provided in a space to be sterilized, the second control circuit is provided at a door of the space to be sterilized, and the third control circuit is provided in the space to be sterilized;
the first control circuit is designed for accessing a first light turning-on signal when there is not an operator in the space to be sterilized and sending a first light turning-on signal to the system control module;
the second control circuit is designed for accessing a second light turning-on signal when the door is closed in the space to be sterilized and sending a second light turning-on signal to the system control module;
the third control circuit is designed to access the third light turning-on signal after an operator leaves the space to be sterilized and closes the door and send a third light turning-on signal to the system control module;
the system control module is used for receiving the first light turning-on signal, the second light turning-on signal and the third light turning-on signal, when the time of receiving the first light turning-on signal is earlier than that of the third light turning-on signal, turning-on drive signal is generated according to the first light turning-on signal, the second light turning-on signal and the third light turning-on signal, and the turning-on drive signal is sent to the switch control module;
the switch control module is used for receiving the turning-on drive signal and controlling the tuning-on of the UV light according to a turning-on drive signal;
the second control circuit is also used for accessing a turning-off signal when the door is opened and sending the light turning-off signal to the system control module;
the system control module is also used for receiving a light turning-off signal, generating turning-off drive signals according to the light turning-off signal and sending the turning-off drive signal to the switch control module; and
the switch control module is used for receiving the turning-off drive signal and controlling the tuning-off of the UV light according to the turning-off drive signal.

17. The man-free sterilization system control device as claimed in claim 16, wherein the switch control module, the system control module and the first control circuit form the independent first control module;
and/or, the third control circuit is an independent second control module.

18. The man-free sterilization system control device as claimed in claim 16, wherein the switch control module is a relay;
and/or, the system control module is a single chip microcomputer control circuit;
and/or, the second control circuit is a magnetic door contact circuit device or a mechanical switch circuit, the door automatically accesses the second light turning-on signal when the door is closed, and the door automatically accesses the light turning-off signal when the door is opened.

19. The man-free sterilization system control device as claimed in claim 16, wherein the first control circuit comprises one or several light turning-on keys, the light turning-on keys provide the first light turning-on signal;
when there is only one light turning-on key, this light turning-on key is used for providing the first light turning-on signal or turning-off signal;
when there are several light turning-on keys, one of these light turning-on keys is used for providing the first light turning-on signal or turning-off signal, and other light turning-on keys are used for setting the light turning-on time.

20. The man-free sterilization system control device as claimed in claim 16, wherein the third control circuit comprises one or several light turning-on keys, the light turning-on keys provide the third light turning-on signal;
when there is only one light turning-on key, this light turning-on key is used for providing the third light turning-on signal or turning-off signal;
when there are several light turning-on keys, one of these light turning-on keys is used for providing the third light turning-on signal or turning-off signal, and other light turning-on keys are used for setting the light turning-on time.

21. The man-free sterilization system control device as claimed in claim 16, wherein the first control circuit comprises the first indicator;
the first indicator is used for reminding the operator of that the first light turning-on signal has been sent when the first indicator goes on;
and/or, the third control circuit comprises the second indicator which is used for reminding the operator of that the third light turning-on signal has been sent when the second indicator goes on.

22. The man-free sterilization system control device as claimed in claim 16, wherein the third control circuit comprises an enabling unit which enables a preset tool to make the third control circuit work normally.

23. The man-free sterilization system control device as claimed in claim 21, wherein the enabling unit is a key switch, and the preset tool is a specific key.

24. The man-free sterilization system control device as claimed in claim 16, wherein the third control circuit comprises a display screen; and
the display screen is used for displaying the work time or the work state of the UV light.

25. The man-free sterilization system control device as claimed in claim 16, wherein the system control module comprises a first extension interface unit, the third control circuit comprises a second extension interface unit, the first extension interface unit and the second extension interface unit are multi-way interfaces, the control signal terminal of the second control circuit connects to the control signal terminal of the second extension interface unit, and the first extension interface unit connects to the second extension interface unit via a control signal cable.

26. The man-free sterilization system control device as claimed in claim 16, wherein the second control circuit comprises several control signal launch units which are provided in different door positions in a serial connection way;
the several control signal launch units are used for sending the second light turning-on signal to the system control module when doors corresponding to all the control signal launch units are closed; and
the several control signal launch units are used for sending the light turning-off signal to the system control module when doors corresponding to any control signal launch units are open.

27. The man-free sterilization system control device as claimed in claim 17, wherein the second control module comprises: the second control device, the third control device, the second signal interface, a switch interface, a fifteenth resistor and a second light emitting diode;
the first terminal of the second control device connects to the first interface of the switch interface, the second terminal of the second control device connects to a fourth interface of the switch interface, the second interface of the switch interface connects to the third interface, the first interface of the switch interface also connects to an eighth interface of the second signal interface, the fourth interface of the switch interface also connects to the first interface of the second signal interface, a fifth interface and a sixth interface of the second signal interface also connect to the first terminal of the third control device, the second terminal of the third control devices is of grounding, the third interface and the fourth interface of the second signal interface connect to a cathode of a second light emitting diode, an anode of the second light emitting diode connects to a first terminal of the fifteenth resistor, a second terminal of the fifteenth resistor connects to a seventh interface of the second signal interface.

28. A control module which is applied in the man-free sterilization system, comprising:
a switch control module, a system control module, a first control circuit and a first control signal extension port, a signal input terminal of the system control module connects to a signal output terminal of the first control circuit and a signal output terminal of a first control signal extension port, a drive signal output terminal of the system control module connects to a drive signal input terminal of the switch control module, and the switch control module connects to a power supply and a UV light;
the first control circuit is designed for accessing a first light turning-on signal when there is not an operator in a space to be sterilized and sending the first light turning-on signal to the system control module;
the first control signal extension port is used for accessing a second light turning-on signal when a door of the space to be sterilized is closed and sending the second light turning-on signal to the system control module;
the first control signal extension port is designed to access a third light turning-on signal after the operator leaves the space to be sterilized and closes the door and send a third light turning-on signal to the system control module;
the system control module is used for receiving the first light turning-on signal, the second light turning-on signal and the third light turning-on signal, when the time of receiving the first light turning-on signal is earlier than that of the third light turning-on signal, turning-on drive signal is generated according to the first light turning-on signal, the second light turning-on signal and the third light turning-on signal, and the turning-on drive signal is sent to the switch control module;
the switch control module is used for receiving the turning-on drive signal and controlling the tuning-on of the UV light according to the turning-on drive signal;
the first control signal extension port is also used for accessing a turning-off signal when the door is opened and sending the light turning-off signal to the system control module;
the system control module is also used for receiving the light turning-off signal, generating turning-off drive signals according to the light turning-off signal and sending the turning-off drive signal to the switch control module; and
the switch control module is used for receiving the turning-off drive signal and controlling the tuning-off of the UV light according to the turning-off drive signal.

29. A man-free sterilization system control circuit comprising:
a power interface, a fuse tube, a voltage-dependent resistor, a rectifier bridge, a relay armature, a relay coil, a first power supply, a second power supply, a first control chip, a second control chip, a first control device, a master single chip microcomputer, a first diode, a fourth diode, a triode, a first resistor, a fourteen resistor, a first signal interface, the first capacitor, a fourth capacitor, a first capacitor, a ninth capacitor, a first inductor, a fourth inductor, a first switch, a fourth switch and a first light emitting diode;
a first interface and a second interface of the power interface connects to a first terminal of a fuse tube, a second terminal of the fuse tube connects to a first terminal of the voltage-dependent resistor and a first AC input terminal of the rectifier bridge, a third interface of the power interface connects to a voltage input terminal of the step-down rectifier unit, a third interface of the power interface connects to a second terminal of the voltage-dependent resistor and a second AC input terminal of the rectifier bridge respectively, a fourth interface of the power interface connects to a second terminal of the relay armature, a positive output terminal of the rectifier bridge connects to the first resistor and the second resistor respectively, a positive output terminal of a rectifier bridge also connects to a first terminal of a first inductor and a first terminal of the first capacitor respectively, a negative output terminal of the rectifier bridge is of grounding, a first terminal of a second capacitor connects to a second terminal of the first resistor and a second terminal of the first inductor, respectively, a second terminal of the first capacitor and the second terminal of the second capacitor are of grounding, a second terminal of the first inductor connects to a first terminal of the fifth capacitor and a first terminal of the fifth resistor respectively, a first terminal of the sixth resistor connects to the second terminal of the fifth capacitor and a second terminal of the fifth resistor respectively, a second terminal of the sixth resistor connects to a cathode of the first diode, a second terminal of the first inductor connects to a first terminal of the second inductor, a fifth through eighth interfaces of the first control chip connect to the anode of the first diode and the second terminal of the second inductor, a second terminal of a second resistor connects to the first terminal of a third resistor, a second terminal of the third resistor connects to a first terminal of the fourth resistor, a first interface of the first control chip connects to a cathode of the second diode and a first terminal of the third capacitor respectively, a second terminal of the fourth resistor connects to a first terminal of the third capacitor, an anode of the second diode connects to a first terminal of the third inductor and a first terminal of a seventh resistor respectively, a second terminal of the seventh resistor connects to a first terminal of the eighth resistor and a first terminal of the sixth capacitor, a second terminal of a third inductor and a second terminal of an eighth resistor are of grounding, a third interface of the first control chip connects to a first terminal of the sixth capacitor, a second terminal of the third capacitor and a second terminal of the sixth capacitor are of grounding, the second interface of the first control chip is of grounding, a fourth interface of the first control chip connects to a first terminal of a ninth resistor, a second terminal of the ninth resistor connects to a first terminal of the seventh capacitor, a second terminal of the ninth resistor is of grounding, a second terminal of the seventh capacitor connects to a first terminal of the fourth inductor, a first terminal of the fourth inductor is of grounding, a second terminal of the fourth inductor connects to an anode of a third diode, a cathode of a third diode connects to a first terminal of a fourth capacitor and a first terminal of a tenth resistor respectively, a cathode of the third diode also connects to the first power supply, a second terminal of the fourth capacitor and a second terminal of the tenth resistor are of grounding; and
the first terminal of the first control device connects to a first light turning-on signal terminal of the master single chip microcomputer, a second terminal of the first control device is of grounding; a first terminal of the relay coil connects to the cathode of the diode, the first terminal of the relay coil also connects to the first power supply, a second terminal of the relay coil connects to an anode of the diode, the second terminal of the relay coil also connects to a collector electrode of the triode, a base electrode of the triode connects to the first terminal of the first resistor, the second terminal of the first resistor connects to a master single chip microcomputer, the first terminal of the first resistor also connects to the first terminal of the second resistor, the second terminal of the second resistor connects to the emitting electrode of the triode, the emitting electrode of the triode is of grounding, a voltage input terminal of the second control chip connects to the first power supply, the voltage output terminal of the second control chip connects to the voltage input terminal of the master single chip microcomputer, the voltage output terminal of the second control chip connects to the first terminal of a thirteenth resistor and the first terminal of the ninth capacitor respectively, the second terminal of the thirteenth resistor connectors to the anode of the first light emitting diode, a cathode of the first light emitting diode connects to the control terminal of the first indicator of the master single chip microcomputer, the first terminal of the ninth capacitor also connects to the first terminal of the fourteenth resistor, a second terminal of the fourteenth resistor connects to the second power supply, a second terminal of the ninth resistor is of grounding, a grounding terminal of the second control chip and a grounding terminal of the master single chip microcomputer are of grounding, a first terminal of the eighth capacitor connects to the first power supply, a second terminal of the eighth capacitor is of grounding, the first interface and the second interface of the first signal interface are of grounding, a third interface and a fourth interface of the first signal interface connects to the signal terminal of a second indicator of the master single chip microcomputer, a fifth interface and a sixth interface of the first signal interface connects to the second light turning-on signal terminal of the master single chip microcomputer, a seventh interface of the first signal interface connects to the second power supply, an eighth interface of the first signal interface connects to the third light turning-on signal terminal of the master single chip microcomputer, the first terminal of the first switch connects to the signal terminal of the a clock of the master single chip microcomputer, a first terminal of the second switch connects to the signal terminal of the second clock of the master single chip microcomputer, a first terminal of the third switch connects to a signal terminal of a third clock of the master single chip microcomputer, a first terminal of a fourth switch connects to a signal terminal of a fourth clock of the master single chip microcomputer, and a second terminal of the first through fourth switches is of grounding.

30. A man-free sterilization system control method which is applied to a man-free sterilization system control device comprising:
a switch control module, a system control module, the first control circuit, the second control circuit and the third control circuit;
the man-free serialization system control method is as follows:
a first control circuit is designed for accessing a first light turning-on signal when there is not an operator in a space to be sterilized and sending the first light turning-on signal to the system control module;
a second control circuit is designed for accessing a second light turning-on signal when a door is closed in the space to be sterilized and sending the second light turning-on signal to the system control module;
a third control circuit is designed to access a third light turning-on signal after the operator leaves the space to be sterilized and closes the door and send a third light turning-on signal to the system control module;
the system control module is used for receiving the first light turning-on signal, the second light turning-on signal and the third light turning-on signal, when the time of receiving the first light turning-on signal is earlier than that of the third light turning-on signal, turning-on drive signal is generated according to the first light turning-on signal, the second light turning-on signal and the third light turning-on signal, and the turning-on drive signal is sent to the switch control module;
the switch control module is used for receiving the turning-on drive signal and controlling the tuning-on of a UV light according to the turning-on drive signal;
a second control circuit is also used for accessing the turning-off signal when the door is opened and sending the light turning-off signal to the system control module;
the system control module is also used for receiving the light turning-off signal, generating turning-off drive signals according to the light turning-off signal and sending the turning-off drive signal to the switch control module; and
the switch control module is used for receiving the turning-off drive signal and controlling the tuning-off of the UV light according to the turning-off drive signal.
